(19) United States
(12) Patent Application Publication
(10) Pub. No.: US 2025/0072884 A1
(43) Pub. Date: Mar. 6, 2025

(54) LATERAL ROW ANCHOR

(71) Applicant: Biomet Manufacturing, LLC, Warsaw, IN (US)

(72) Inventor: Nathan A. Winslow, Scottsdale, AZ (US)

(21) Appl. No.: 18/819,756

(22) Filed: Aug. 29, 2024

Related U.S. Application Data
(60) Provisional application No. 63/536,589, filed on Sep. 5, 2023.

(51) Int. Cl. A61B 17/04

(52) U.S. Cl. CPC A61B 17/0469; A61B 2017/0409; A61B 2017/0414

(57) ABSTRACT

A system may include a proximal body defining a shaft. A system may include a distal body for insertion into the proximal body shaft, the distal body comprising: a square post, wherein an internal surface of the square post is threaded; and a suture slot in having at least one surface with serrations thereon. A system may include a screw at least partially encapsulated within the proximal body and extending into the shaft, wherein the screw is threaded to match the internal surface of the threaded square post. A system may include the screw actuatable to move the distal body into the shaft by rotation of the screw.

### CLAIM OF PRIORITY

This application claims the benefit of U.S. Provisional Patent Application Ser. No. 63/536,589, filed on Sep. 5, 2023, the benefit of priority of which is claimed hereby, and which is incorporated by reference herein in its entirety.

### BACKGROUND

Various implant systems have been created for repairing soft-tissue damage in different surgical contexts. As an example, suture anchors have been utilized to repair tears to a patient's rotator cuff, a patient's labrum in the shoulder or hip, or a tear to a patient's meniscus in the knee.

In some cases, the suture anchor is driven into a pre-drilled bone hole, and suture is passed through the particular tissue in order to tension the suture construct and draw the tissue back into its normal anatomical position against the bone. At the end of the repair, a knot can be used to assist in drawing the torn soft tissue back to its anatomically-correct position and/or to secure the tissue in position.

The implantation of sutures with anchors can be difficult. For example, tying knots in sutures can be very time consuming and difficult to perform, particularly inside the joint space. As a result, the cost of the procedure can be increased, and the capacity of the surgeon can be limited. Furthermore, the strength of the repair can be limited by the strength of the knot. The use of knots can result in a number of issues for the patient such as knot migration, knot stack irritation, and knot loosening.

Knotless suture anchors have also been developed, but such anchors can suffer from deficiencies in securing the suture relative to the anchor. Often, a friction-lock is used in which the suture is pressed against the anchor in an attempt to secure the suture relative to the anchor. Such constructs can suffer from slippage and loss of tension between the suture and the anchor at the point of the friction-lock.

### SUMMARY OF THE DISCLOSURE

In an example, the techniques described herein relate to an anchoring system for securing suture to bone, the anchoring system including: a proximal body defining a shaft; a distal body for insertion into the proximal body shaft, the distal body including: a square post, wherein an internal surface of the square post is threaded; and a suture slot in having at least one surface with serrations thereon; and a screw at least partially encapsulated within the proximal body and extending into the shaft, wherein the screw is threaded to match the internal surface of the threaded square post, the screw actuatable to move the distal body into the shaft by rotation of the screw.

In an example, the techniques described herein relate to an anchoring system for securing suture to bone, the anchoring system including: a proximal body defining a shaft, the proximal body having a first suture slot; a distal body for insertion into the proximal body shaft, the distal body including: a square post, wherein an internal surface of the square post is threaded; and a second suture slot in having at least one surface with serrations thereon, wherein in a first mode, the first suture slot and the second suture slot are aligned, and wherein in a second mode, where the square post is fully inserted into the shaft, the first suture slot and the second suture slot are misaligned.

In an example, the techniques described herein relate to a method of tissue repair, the method including: embedding a first portion of an anchoring system in bone, the first portion of the anchoring system defining a cavity for receiving a square post; aligning a second portion of the anchoring system over the first portion of the anchoring system, the second portion of the anchoring system having the square post; threading one or more sutures through a suture slot in the second portion of the anchoring system, and tightening the one or more sutures by moving the first portion of the anchoring system and the second portion of the anchoring system together.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1K illustrate various views and schematic diagrams of a lateral row anchor anchoring system with a proximal body and a distal body in an example.

FIGS. 2A-2B depict a lateral row anchor anchoring system with sutures in an example.

FIGS. 3A-3B depict a lateral row anchor anchoring system with dual circular suture slots in an example.

FIG. 4 depicts a lateral row anchor anchoring system with wings in an example.

FIG. 5 depicts a method of using a lateral row anchor anchoring system such as those described above.

### DETAILED DESCRIPTION

The present disclosure describes, among other things, a lateral row anchor method leveraging a square post and suture slot in combination with a screw mechanism to provide alterable and secure lateral row sutures. The anchor method and accompanying anchoring systems discussed herein are for use with knotless sutures for use in lateral row procedures.

Knotless sutures have a variety of benefits. Specifically, the anchoring system and methods discussed herein allow for placement of an anchor and suture in the bone which allows for tightening of the sutures and prevention of loosening. The anchoring system and methods discussed herein also allow for re-tensioning of sutures as desired. Additionally, the anchoring system and methods discussed herein include an anchor with three suture fixation points, allowing for a stronger suture and anchor structure overall.

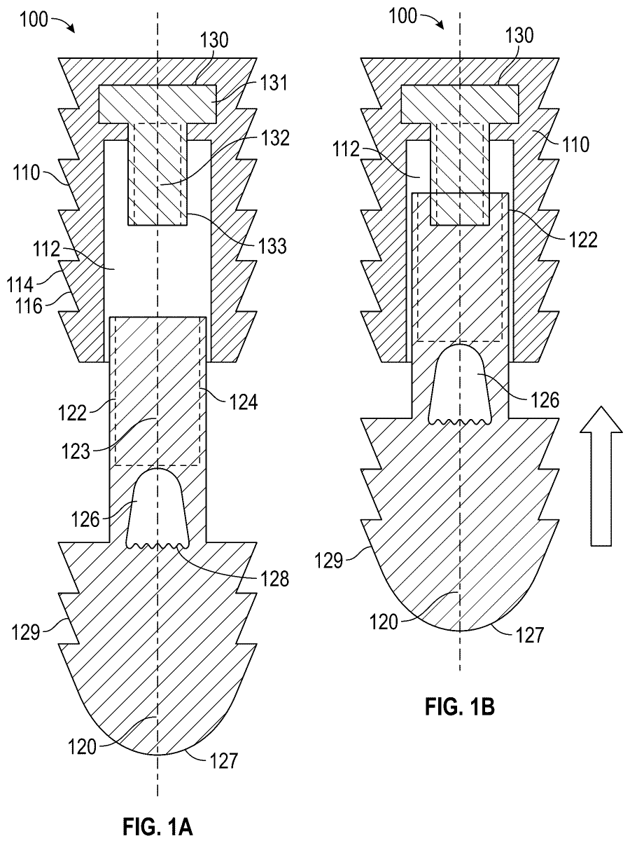

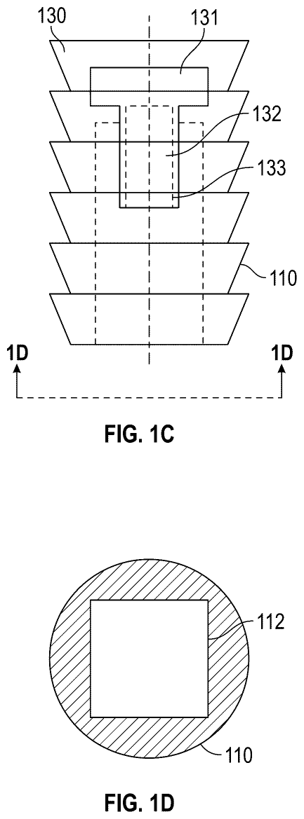

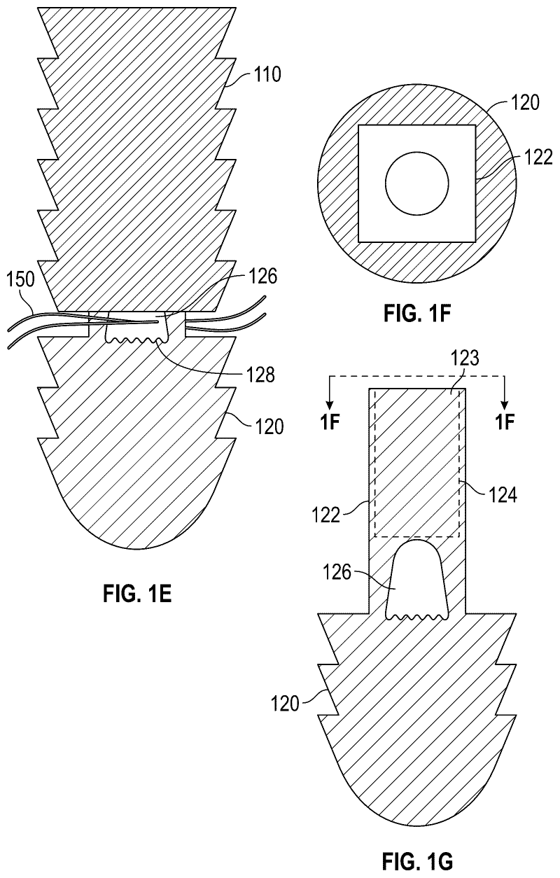

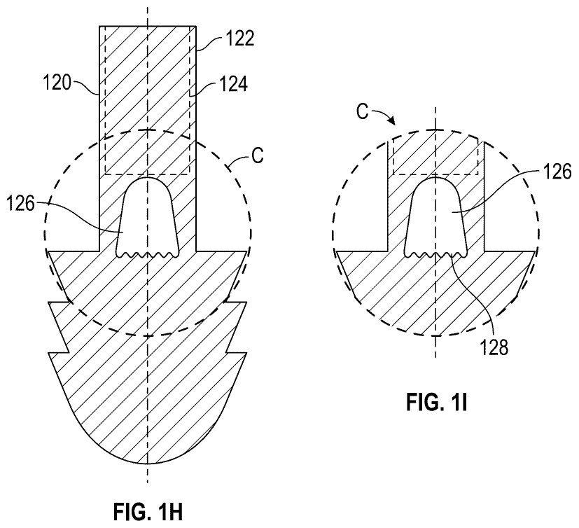

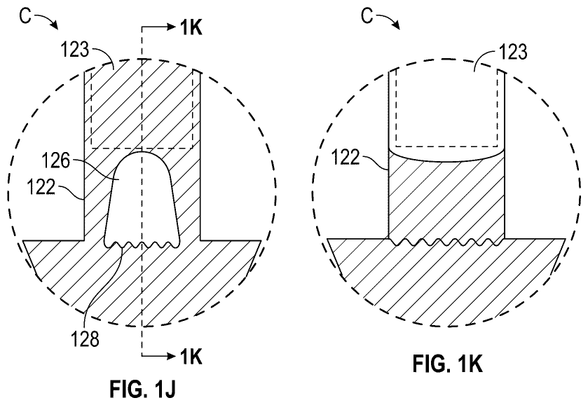

FIGS. 1A-1K illustrate various views and schematic diagrams of a lateral row anchor anchoring system 100 with a proximal body 110 and a distal body 120. The proximal body 110 can include a shaft 112. The distal body 120 can include a square post 122 with a threaded surface 124, and a suture slot 126 with serrations 128 thereon. The lateral row anchor anchoring system 100 can further include a screw 130 at least partially encapsulated within the proximal body 110 and extending into the shaft 112. The screw 130 can be threaded to match the threaded surface 124 of the square post 122. The screw 130 can be actuatable to move the distal body 120 into the shaft 112 by rotation of the screw 130.

The lateral row anchor anchoring system 100 can be used, for example, to secure sutures in a lateral row. For example, the lateral row anchor anchoring system 100 can be used to secure tissue to bone along the lateral row. The lateral row anchor anchoring system 100 can be a knotless system, such that the sutures do not require knots.

The proximal body 110 can be the piece typically used closer to the operator, such as during tightening of the lateral row anchor anchoring system 100. The proximal body 110 can host the shaft 112 and the screw 130. In an example, the proximal body 110 can have an external surface 114 with serrations 116. The serrations 116 can help secure the proximal body 110 in the bone. The serrations 116 can act as anchor points for the lateral row anchor anchoring system 100.

The shaft 112 can be a shaft extending within the proximal body 110. The shaft 112 can have a square cross-section to prevent rotation when the proximal body 110 is joined with the distal body 120. This can help prevent movement and tissue damage when the sutures are inserted. The shaft 112 can be sized and shaped for receipt of the square post 122.

The distal body 120 can serve as an anchor point for the lateral row anchor anchoring system 100. The distal body 120 can be shaped and sized for embedding in bone. The distal body 120 can include the suture slot 126 and the square post 122. The distal body 120 can have an end 127 that includes serrations 129. The serrations 129 can allow for anchoring of the distal body 120 in bone or other tissue. The serrations 129 can partially or fully cover the end 127.

The square post 122 can extend proximally from the end 127 of the distal body 120. The square post 122 can host the suture slot 126. The square post 122 can be sized and shaped for insertion into the shaft 112. The square post 122 can include a cavity 123 with a threaded surface 124. The cavity 123 can be sized and shaped to mate with the screw 130. The threaded surface 124 can match with threads of the screw 130.

The suture slot 126 can be, for example, between the end 127 and an internal surface of the square post. In an example, the suture slot 126 can be a half-oval shape, a half-circular shape, a circular shape, and oval shape, a polygonal shape, or another appropriate shape for receipt of one or more suture threads or strands. The suture slot 126 can further include one or more surfaces having serrations 128. The serrations 128 can be situated to aid in securing one or more suture threads therein, such as to reduce slippage.

The internal surface of the square post cavity 123 and the suture slot 126 do not overlap with the distal body 120. As such, this allows securing one or more suture strands within the suture slot 126, but not within the cavity 123.

The screw 130 can be situated within the proximal body 110. In some cases, the screw 130 can including a head 131 embedded in the proximal body 110, and an extension 132 extending outwards from the head 131 into the shaft 112. The extension 132 can include threads 133 that mate with the threaded surface 124 of the cavity 123.

Alternatively, the screw 130 can have threads on its shaft 133 and on the outside diameter of the head 131. This would allow the screw 130 to be threaded into the proximal body 110 through a threaded hole at the end of the proximal body 110. Once the screw 130 had been sufficiently inserted it would be in a recess where the screw 130 could turn and be captured by the thread. In such a case, a user could access the screw 130 through a hole to tighten the screw 130 into the threaded surface 124.

FIG. 1A and FIG. 1B show side views of the lateral row anchor anchoring system 100 include both the proximal body 110 and the distal body 120. In FIG. 1B, the square post 122 of the distal body 120 is being inserted into the shaft 112. FIG. 1C shows a close up side-view of the screw 130 in the proximal body 110. FIG. 1D depicts a top-down cross section of the proximal body 110, such that the square cross section of the shaft 112 can be seen.

FIG. 1E depicts sutures 150 in the suture slot 126. Here, the distal body 120 is being inserted into the proximal body 110, such that the square post 122 is inserted into the shaft 112 while the sutures 150 are in the suture slot 126. The serrations 128 can hold the sutures 150 in place. The sutures 150 can be captured between the bottom of the suture slot 126 and the bottom of the proximal body 110.

FIGS. 1F-1G depict the square post 122 on the distal body 120. FIG. 1G shows a side cross-section view of the square post 122 on the distal body 120, while FIG. 1F depicts a top-down view of the square post 122.

FIGS. 1H-1K depict a closer look the suture slot 126 in the distal body 120. The suture slot 126 can have a flat bottom internal surface to pass through a large number of sutures. The internal surface can host the serrations 128, which can for example include teeth that can grip the sutures when the distal body 120 is fully pulled into the proximal body 110. Tension can be adjusted by releasing the suture capture. FIG. 1K depicts a side cross-sectional view of the suture slot 126.

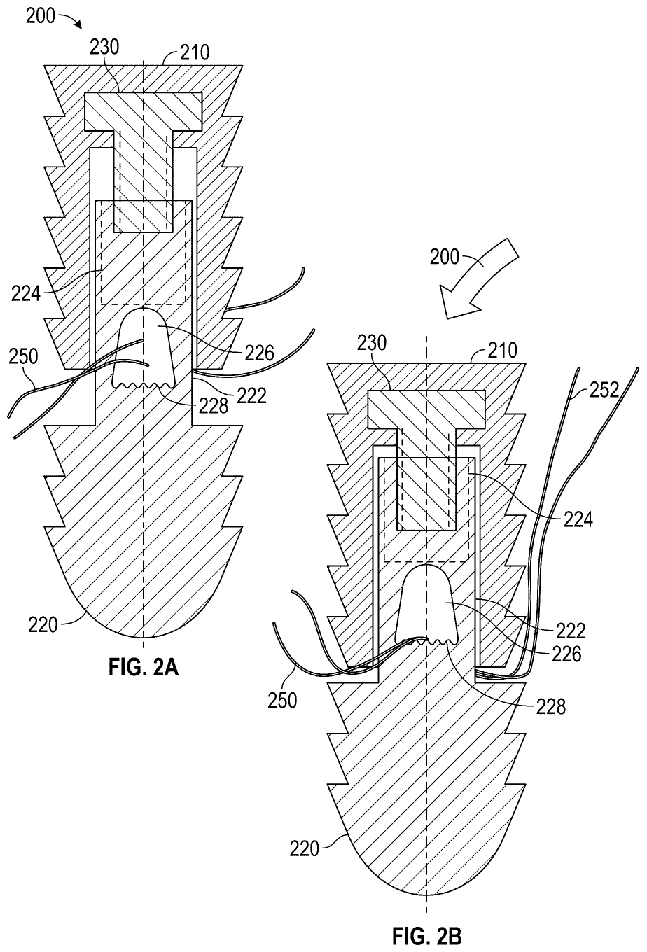

FIGS. 2A-2B depict a lateral row anchor anchoring system 200 with sutures 250. Similar to the lateral row anchor anchoring system 100 discussed above, the lateral row anchor anchoring system 200 can have a proximal body 210 and a distal body 220. The proximal body 210 can include a shaft 212. The distal body 220 can include a square post 222 with a threaded surface 224, and a suture slot 226 with serrations 228 thereon. The lateral row anchor anchoring system 200 can further include a screw 230 at least partially encapsulated within the proximal body 210 and extending into the shaft 212. The screw 230 can be threaded to match the threaded surface 224 of the square post 222. The screw 230 can be actuatable to move the distal body 220 into the shaft 212 by rotation of the screw 230.

Here, positioning the suture slot 226 more proximally on the square post 222 can allow the user to tension repair strands 252 by pulling them into the anchor, shown FIG. 2B. Additional tension can be used, or strands can be preset. For example, the proximal body 210 can include a full cylinder, such that the suture strands are trapped between the axial walls of square post 222 and proximal body 210, as well as between the radial walls at the end of the proximal body 210. Thus, sutures are trapped along a bent path rather than being flat. Additionally, this feature can allow for the sutures to be held in place on the anchor to allow for tensioning or positioning before being finally tightened down.

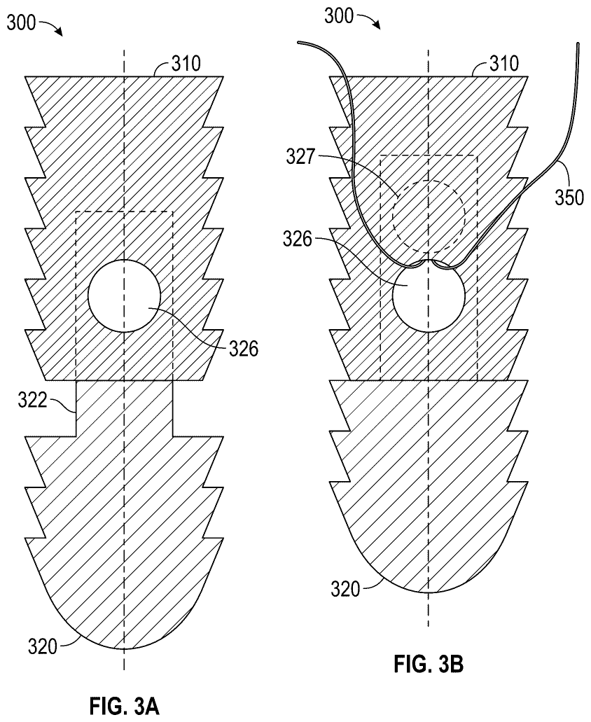

FIGS. 3A-3B depict a lateral row anchor anchoring system 300 with dual circular suture slots 326, 327. Similar to the lateral row anchor anchoring system 100 discussed above, the lateral row anchor anchoring system 300 can have a proximal body 310 and a distal body 320. The proximal body 310 can include a shaft 312. The distal body 320 can include a square post 322 that slots into the shaft 312.

Here, the proximal body 310 can include suture slot 327, while the distal body 320 can include the suture slot 326. The two suture slots 326, 327, can be circular in shape. When the proximal body 310 and the distal body 320 are not fully inserted, the circular suture slot 326 and the circular suture slot 327 can be aligned, as shown in FIG. 3A. Here, sutures 350 can be inserted. The two suture slots 326, 327, can be used, for example, for repair strands.

As shown in FIG. 3B, when the proximal body 310 and the distal body 320 fully or partially inserted, the suture slots 326, 327, can be misaligned, pulling the sutures 350 into the lateral row anchor anchoring system 300. Actuation of the lateral row anchor anchoring system 300 can pull the distal body 320 into the proximal body 310 and capture the sutures 350 between the walls of shaft 312 and the sutures slots 326, 327.

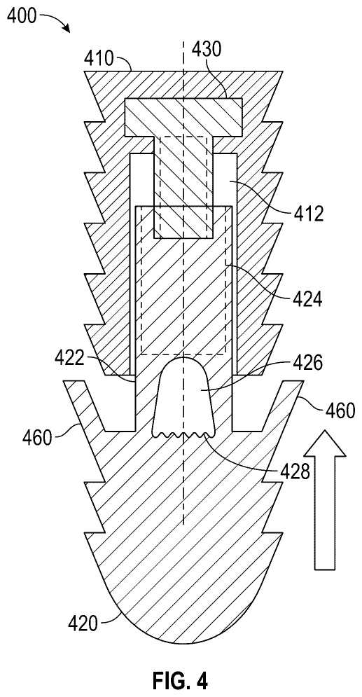

FIG. 4 depicts a lateral row anchor anchoring system 400 with wings 460. Similar to the lateral row anchor anchoring system 100 discussed above, the lateral row anchor anchoring system 400 can have a proximal body 410 and a distal body 420. The proximal body 410 can include a shaft 412. The distal body 420 can include a square post 422 with a threaded surface 424, and a suture slot 426 with serrations 428 thereon. The lateral row anchor anchoring system 400 can further include a screw 430 at least partially encapsulated within the proximal body 410 and extending into the shaft 412. The screw 430 can be threaded to match the threaded surface 424 of the square post 422. The screw 430 can be actuatable to move the distal body 420 into the shaft 412 by rotation of the screw 430.

Here, the wings 460 can be on the distal body 420. The wings 460 can hit the proximal body 410 during insertion. When the distal body 420 and the proximal body 410 are joined the wings 460 flare outward. This allows for lateral row anchor anchoring system 400 to resist being pulled out from bone. In particular, for lateral row anchors, this provides additional support and anchoring. Moreover, the wings 460 can help hold the distal body 420 in place while the screw is being tightened to the proximal body 410 . . . . Wings 460 can be used with any of the examples described herein.

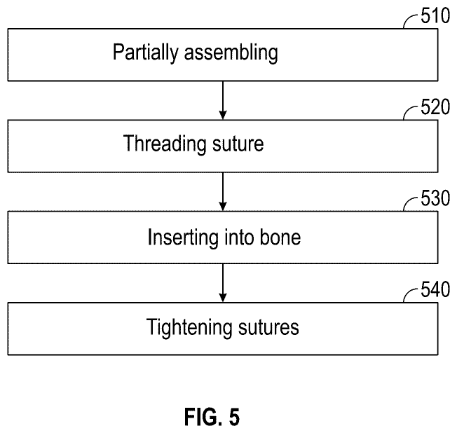

FIG. 5 depicts a method 500 of using a lateral row anchor anchoring system such as those described above.

In method 500 a distal anchor is partially assembled (block 510). For example, the proximal anchor is aligned. The sutures are threaded into the suture hole (block 520). The device is anchored in bone (block 530). The proximal anchor and the distal anchor are then tightened towards each other, such that the square post with the suture hole goes into a cavity on the proximal anchor (block 540). This tightening can be done by the tightening of a screw.

The method 500 is illustrated in part through FIG. 1. At FIG. 1, the proximal and distal section are partially assembled first. For example, the screw is partially screwed into the distal shaft. Then, the suture is passed through device. The device is then inserted into bone. The suture is then tensioned. The screw is turned to pull the distal section to the proximal section, thereby locking suture in place. A similar approach is shown in FIGS. 2-4, however, the suture is partially tensioned. Turning the screw locks the suture in place and additionally tensions the suture.

### Various Notes & Examples

Example 1 is an anchoring system for securing suture to bone, the anchoring system comprising: a proximal body defining a shaft; a distal body for insertion into the proximal body shaft, the distal body comprising: a square post, wherein an internal surface of the square post is threaded; and a suture slot in having at least one surface with serrations thereon; and a screw at least partially encapsulated within the proximal body and extending into the shaft, wherein the screw is threaded to match the internal surface of the threaded square post, the screw actuatable to move the distal body into the shaft by rotation of the screw.

In Example 2, the subject matter of Example 1 optionally includes wherein the shaft is shaped for receipt of the square post.

In Example 3, the subject matter of any one or more of Examples 1-2 optionally include wherein the proximal body comprises an external surface having a plurality of serrations.

In Example 4, the subject matter of any one or more of Examples 1-3 optionally include wherein the distal body further comprises an end configured for embedding in bone.

In Example 5, the subject matter of Example 4 optionally includes wherein the square post extends proximally from the end.

In Example 6, the subject matter of any one or more of Examples 4-5 optionally include wherein the end comprises a plurality of serrations.

In Example 7, the subject matter of any one or more of Examples 4-6 optionally include wherein the suture slot is between the end and the internal surface of the square post.

In Example 8, the subject matter of any one or more of Examples 1-7 optionally include wherein the suture slot comprises a half-oval shape.

In Example 9, the subject matter of any one or more of Examples 1-8 optionally include wherein the suture slot comprises a circular shape.

In Example 10, the subject matter of any one or more of Examples 1-9 optionally include wherein the internal surface of the square post and the suture slot do not overlap.

In Example 11, the subject matter of any one or more of Examples 1-10 optionally include one or more wings extending radially outward from the distal body.

In Example 12, the subject matter of Example 11 optionally includes wherein the one or more wings comprise two lateral wings on the distal body.

In Example 13, the subject matter of any one or more of Examples 11-12 optionally include wherein the one or more wings are distal of the square post.

In Example 14, the subject matter of any one or more of Examples 11-13 optionally include wherein the one or more wings comprise a ring around the distal body.

Example 15 is an anchoring system for securing suture to bone, the anchoring system comprising: a proximal body defining a shaft, the proximal body having a first suture slot; a distal body for insertion into the proximal body shaft, the distal body comprising: a square post, wherein an internal surface of the square post is threaded; and a second suture slot in having at least one surface with serrations thereon, wherein in a first mode, the first suture slot and the second suture slot are aligned, and wherein in a second mode, where the square post is fully inserted into the shaft, the first suture slot and the second suture slot are misaligned.

In Example 16, the subject matter of Example 15 optionally includes wherein the first suture slot and the second suture slot are circular.

In Example 17, the subject matter of any one or more of Examples 15-16 optionally include wherein the first suture slot and the second suture slot comprises serrations therein.

Example 18 is a method of tissue repair, the method comprising: receiving a first portion of an anchoring system, the first portion of the anchoring system defining a cavity for receiving a square post; aligning a second portion of the anchoring system over the first portion of the anchoring system, the second portion of the anchoring system having the square post; threading one or more sutures through a suture slot in the second portion of the anchoring system; embedding the anchoring system in bone; and tightening the one or more sutures by moving the first portion of the anchoring system and the second portion of the anchoring system together.

In Example 19, the subject matter of Example 18 optionally includes wherein tightening comprises inserting the square post of the second portion of the anchoring system.

In Example 20, the subject matter of any one or more of Examples 18-19 optionally include wherein tightening comprises using a screw threaded to a portion of the square post.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An anchoring system for securing suture to bone, the anchoring system comprising:
a proximal body defining a shaft;
a distal body comprising:
a square post for insertion into the proximal body shaft, wherein an internal surface of the square post is threaded; and
a suture slot therein; and
a screw at least partially encapsulated within the proximal body and extending into the shaft, wherein the screw is threaded to match the internal surface of the threaded square post,
the screw actuatable to move the distal body into the shaft by rotation of the screw.

2. The anchoring system of claim 1, wherein the shaft is shaped for receipt of the square post.

3. The anchoring system of claim 1, wherein the proximal body comprises an external surface having a plurality of serrations.

4. The anchoring system of claim 1, wherein the distal body further comprises an end configured for embedding in bone, wherein the square post extends proximally from the end.

5. The anchoring system of claim 1, wherein the suture slot has at least one surface with serrations.

6. The anchoring system of claim 4, wherein the end comprises a plurality of serrations.

7. The anchoring system of claim 4, wherein the suture slot is between the end and the internal surface of the square post cavity.

8. The anchoring system of claim 4, wherein the suture slot is proximate a top of the end.

9. The anchoring system of claim 4, wherein the suture slot is spaced proximally from a top of the end.

10. The anchoring system of claim 1, wherein the suture slot comprises a half-oval shape or a circular shape.

11. The anchoring system of claim 1, wherein the internal surface of the square post cavity and the suture slot do not overlap.

12. The anchoring system of claim 1, further comprising one or more wings extending radially outward from the distal body.

13. The anchoring system of claim 12, wherein the one or more wings are distal of the square post.

14. The anchoring system of claim 12, wherein the one or more wings comprise a ring around the distal body.

15. An anchoring system for securing suture to bone, the anchoring system comprising:
a proximal body defining a shaft, the proximal body having a first suture slot;
a distal body for insertion into the proximal body shaft, the distal body comprising:
a square post, wherein an internal surface of the square post is threaded; and
a second suture slot in having at least one surface with serrations thereon,
wherein in a first mode, the first suture slot and the second suture slot are aligned, and
wherein in a second mode, where the square post is inserted into the shaft, the first suture slot and the second suture slot are misaligned.

16. The anchoring system of claim 15, wherein the first suture slot and the second suture slot are circular.

17. The anchoring system of claim 15, wherein the first suture slot and the second suture slot comprises serrations therein.

18. A method of tissue repair, the method comprising:
receiving a first portion of an anchoring system, the first portion of the anchoring system defining a cavity for receiving a square post;
aligning a second portion of the anchoring system over the first portion of the anchoring system, the second portion of the anchoring system having the square post;
threading one or more sutures through a suture slot in the second portion of the anchoring system;
embedding the anchoring system in bone; and
tightening the one or more sutures by moving the first portion of the anchoring system and the second portion of the anchoring system together.

19. The method of claim 18, wherein tightening comprises inserting the square post of the second portion of the anchoring system or using a screw threaded to a portion of the square post.

20. The method of claim 18, further comprising pre-tensioning the one or more sutures.
